(19) United States
(12) Patent Application Publication
(10) Pub. No.: US 2024/0293003 A1
(43) Pub. Date: Sep. 5, 2024

(54) DEVICE AND METHOD FOR ENHANCED VISUALIZATION OF THE SMALL INTESTINE

(71) Applicant: Neptune Medical Inc., Burlingame, CA (US)

(72) Inventors: Alexander Q. TILSON, Burlingame, CA (US); Garrett J. GOMES, Pleasant Hill, CA (US); Stephen J. MORRIS, Sunnyvale, CA (US); Eugene F. DUVAL, Menlo Park, CA (US); Adam S. WIGGINTON, Sunnyvale, CA (US)

(21) Appl. No.: 18/660,205

(22) Filed: May 9, 2024

Related U.S. Application Data
(63) Continuation of application No. 17/057,240, filed on Nov. 20, 2020.
(60) Provisional application No. 62/678,857, filed on May 31, 2018.

(51) Int. Cl. A61B 1/00; A61B 1/04

(52) U.S. Cl. CPC A61B 1/00085; A61B 1/00148; A61B 1/00158; A61B 1/041

(57) ABSTRACT

A device for distending a body lumen for enhanced visualization with a capsule endoscope includes a plurality of struts configured to extend from the capsule endoscope, a retention mechanism configured to attach the plurality of struts to the capsule endoscope, and a trigger configured to release the retention mechanism.

### CROSS REFERENCE TO RELATED APPLICATIONS

This application is a continuation of U.S. patent application Ser. No. 17/057,240, filed Nov. 20, 2020, now U.S. Patent Application Publication No. 2021/0137366, which is a national phase application under 35 U.S.C. 371 of International Patent Application No. PCT/US2019/034881, filed on May 31, 2019, now International Publication No. WO 2019/232354, which claims priority to U.S. Provisional Patent Application No. 62/678,857, filed May 31, 2018, each of which is incorporated by reference herein.

This application may also be related to International Application No. PCT/US17/47591, filed Aug. 18, 2017, the entirety of which is incorporated by reference herein.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Endoscopy, the study or examination of the inside of the body, is a common procedure performed by gastroenterologists inside of the gastrointestinal (GI) tract. It is typically accomplished with long, slender endoscopes, either rigid or flexible.

For typical endoscopy, visualization of the GI tract is significantly enhanced by insufflation, i.e., the release of pressurized gas (air or CO2). Because collapsed tissue inhibits visualization, insufflation is used to expand or distend the GI tract so that it can be fully visualized. Without such inflation, endoscopy moves through only a portion of an often-flattened lumen, failing to image significant parts of the lumen. As a result, incomplete evaluation of the bowel is common when insufflation is not used. Luminal distention is therefore often critical for endoscopy for better visualization, detection, and the efficacy of the entire examination. FIGS. 1A-1E shows the advantages of insufflation distention during a typical endoscopy procedure. FIG. 1A shows the intestine in its deflated state with no markers visible. FIG. 1B shows that with just 50 mL of insufflation, 4 of the 9 markers become visible. FIG. 1C shows insufflation at 200 mL. FIG. 1D shows insufflation at 450 mL. FIG. 1E shows the intestine fully inflated at 1500 mL.

However, traditional endoscopy is invasive, usually requires anesthesia, and can be very difficult to move through the small intestine. Recently, there has been commercial success for a distinctly new class of devices known as capsule endoscopes (CE) (including pill cameras, pillcams, wireless capsule endoscopes, or video capsule endoscopes (VCE)). Capsule endoscopies have been revolutionary for gastroenterology, enabling diagnostic procedures that are non-invasive, do not require anesthesia, and provide visualization of anatomies that were previously poorly interrogated, including the small intestine. With capsule endoscopy, manufacturers have gone to great lengths to improve visualization, adding ever-more cameras, side-view cameras, rotating cameras, cameras with a widening field of view, or finer resolution, an increased quantity of more powerful LEDs, faster data transfer rates, and radically higher frame rates. However, none of these improvements are meaningful or helpful if the lumen is flattened and/or covers the lens.

For capsule endoscopy, luminal expansion techniques are currently not available. As such, the procedure's visualization, and thus its entire diagnostic yield and efficacy, is significantly limited. FIGS. 2A-2D show exemplary instances in which the field of view with a capsule endoscope can be obscured. FIG. 2A, for example, shows the lumen 200 collapsed around the capsule endoscope 222. FIG. 2B shows scope 222 oriented towards a fold in the wall of the lumen 200. FIG. 2C shows localized immersive mucosal contact of the lumen 200 with the scope 222. Finally, FIG. 2D shows deep folds in the lumen 200, which can hinder visualization with the scope 222. These exemplary scenarios can result in occlusion of the lens, a view of only the closed tissue, and/or an impartial view caused by blood, debris, or tissue interfering with the lens. As a result, typical capsule endoscope diagnostic efficacy rates are sub-par, estimated at only around 50%. Despite patient experience advantages relative to traditional endoscopic devices, these suboptimal rates have prevented the devices from reaching their potential.

Studies have been performed with capsule endoscopes that release gas into the gastrointestinal tract for insufflation, and the results show radically improved visualization. Gas release in such studies was accomplished, for example, through the release of pressurized air or as the by-product of a chemical reaction. However, storing and methodically releasing pressurized air aboard a capsule in the gastrointestinal tract is problematic. Excessive localized gas release can cause patient discomfort. Chemical reactions struggle with heat, biocompatibility, foaming and bubbles, longevity, and adequate volume.

Capsule endoscopes including built-in radial extensions have been proposed as a means of making the device more lumen-centric to improve imaging, but these structures do not serve to adequately tent small intestine tissue, as the small intestine tissue is very thin, soft, and compliant and tends to fold over onto the lens of the scope.

A device for use with a capsule endoscope that addresses some or all of these problems is thus desired.

### SUMMARY OF THE DISCLOSURE

In general, in one embodiment, a device for distending a body lumen for enhanced visualization with a capsule endoscope includes a plurality of struts configured to extend from the capsule endoscope, a retention mechanism configured to attach the plurality of struts to the capsule endoscope, and a trigger configured to release the retention mechanism.

This and any other embodiments can include one or more of the following features. The retention mechanism can be a retention strap. The retention strap can be configured to extend around proximal ends of the plurality of struts and the capsule endoscope. The capsule endoscope can include a retention strap groove, and the retention strap can be configured to sit within the retention strap groove. The retention strap can include suture, string, wire, filament or braid. The trigger can be a fuse wire. The fuse wire can be configured to corrode to release the retention mechanism. The capsule endoscope can include an aperture therein that allows fluid to pass therethrough to corrode the fuse wire. The device can further include a power supply attached to the fuse wire. The battery can be configured to accelerate corrosion of the fuse wire. The fuse wire can be a galvanic time release wire. The fuse wire can include a plated cathode and an unplated anode. The plated cathode can include silver. The fuse wire can include magnesium. The retention mechanism can be a retention strap. The fuse wire can be configured to corrode to release the retention strap. The trigger can be attached to the capsule endoscope. The plurality of struts can be configured to meet at an apex. The apex can be positioned a set axial distance away from the camera lens. The apex can include at least one pin around which the struts are wound. The device can further include a second trigger configured to release the plurality of struts from one another. The second trigger can include a corrodible element.

In general, in one embodiment, a device is configured to distend a body lumen for enhanced visualization with a capsule endoscope that includes a camera lens. The device includes a plurality of struts, a retention strap, and a fuse wire. The plurality of struts are configured to extend from the capsule endoscope and meet at an apex with the apex positioned a set axial distance away from the camera lens. The retention strap is configured to extend around the plurality of struts and the capsule endoscope to attach to the capsule endoscope. The fuse wire is configured to release the retention strap.

In general, in one embodiment, a device is configured to distend a body lumen for enhanced visualization with a capsule endoscope that includes a camera lens. The device includes a plurality of struts and a fuse wire. The plurality of struts are configured to attach to and extend from the endoscope and meet at an apex with the apex positioned a set axial distance away from the camera lens. The fuse wire is configured to release the plurality of struts from the endoscope when activated.

In general, in one embodiment, a method of enhancing visualization of a body lumen includes: positioning a device including a capsule endoscope and a plurality of struts attached thereto into a body lumen; expanding the plurality of struts of the device within the body lumen such that the plurality of struts extend away from a camera lens of the capsule endoscope and meet at an apex that is positioned a set axial distance away from the camera lens; imaging with the camera lens into the body lumen; and using a magnetic force to pull the device through a pylorus.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1E show the intestine at various stages of insufflation.

FIGS. 2A-2D are schematics showing exemplary instances in which the field of view with a capsule endoscope can be obscured.

FIG. 3 shows a capsule endoscope with an exemplary luminal expansion device attached to the endoscope with a retention strap.

FIGS. 4A-4B show the luminal expansion device of FIG. 3 with a fuse wire.

FIGS. 5A-5B show the luminal expansion device of FIG. 3 with a groove for the retention strap.

FIGS. 6A-6B show an exemplary distal apex for the luminal expansion device of FIG. 3.

FIG. 7 shows a luminal expansion device with a tug used inside of the gastrointestinal tract.

FIG. 8 shows an exemplary luminal expansion device with a lens at the distal apex.

FIGS. 9A-9C show an exemplary luminal expansion device with a sphere at the distal apex.

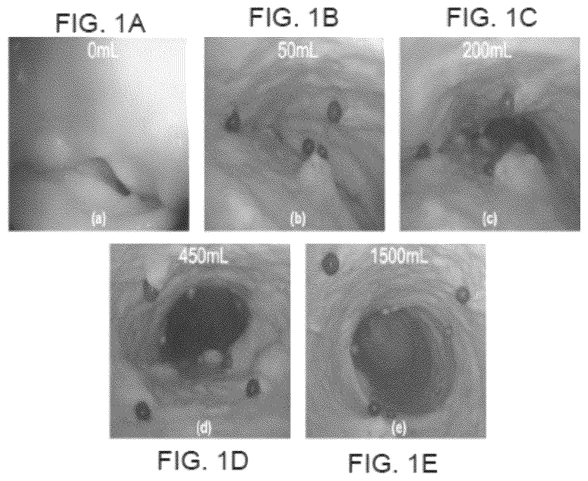

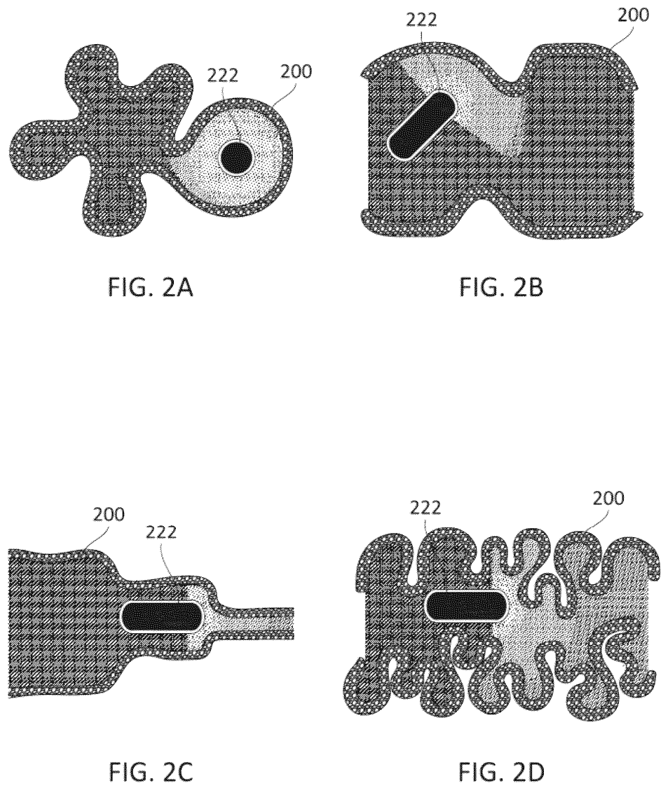

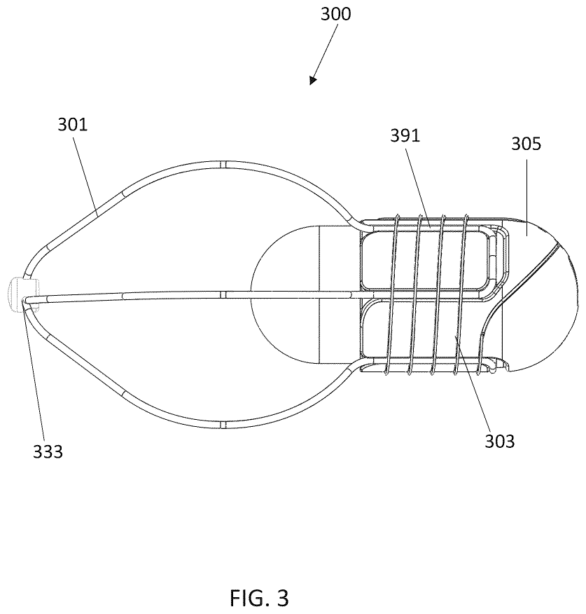

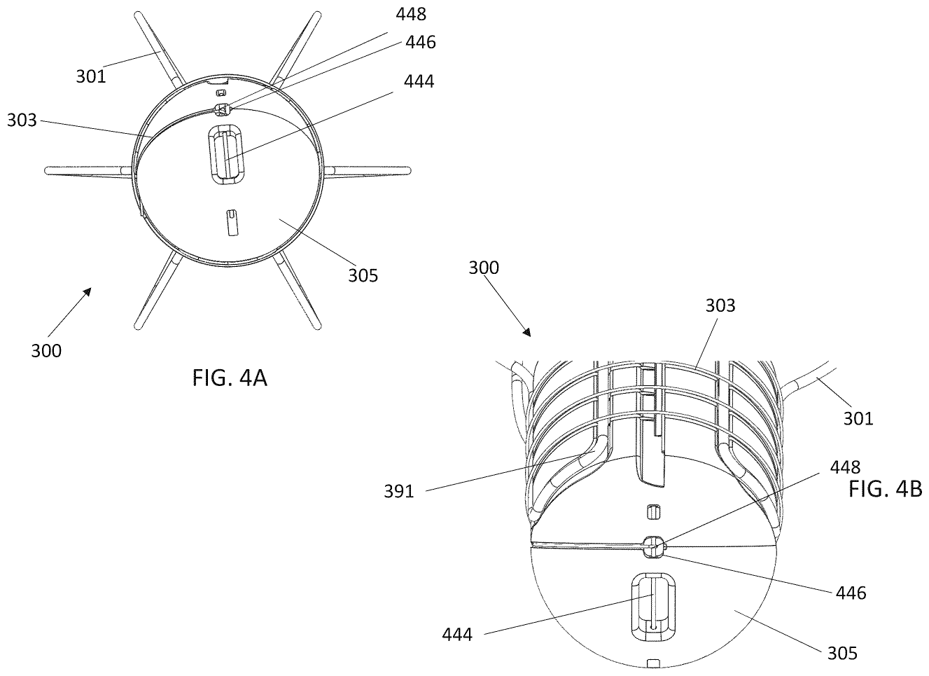

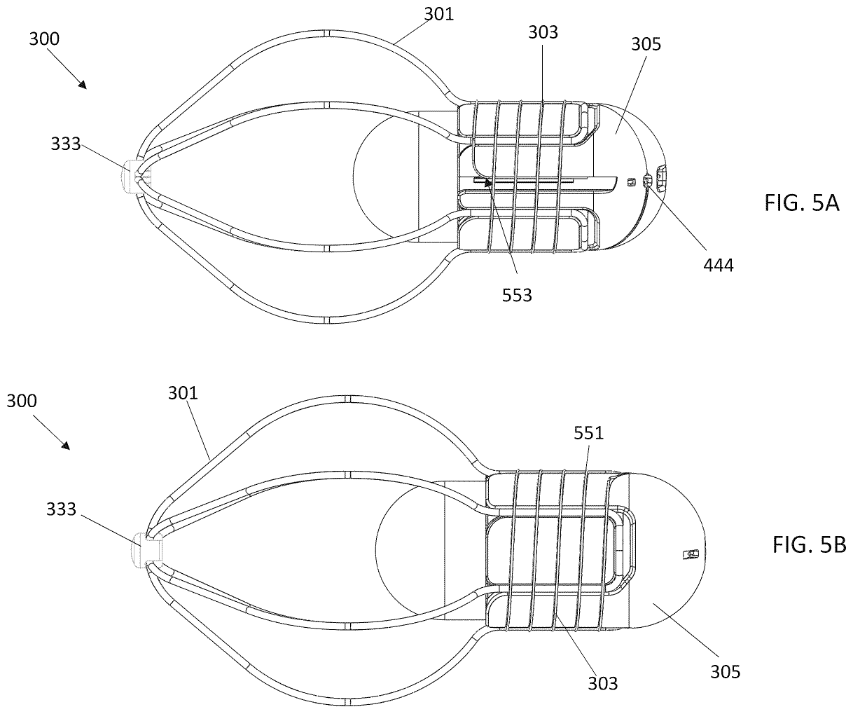

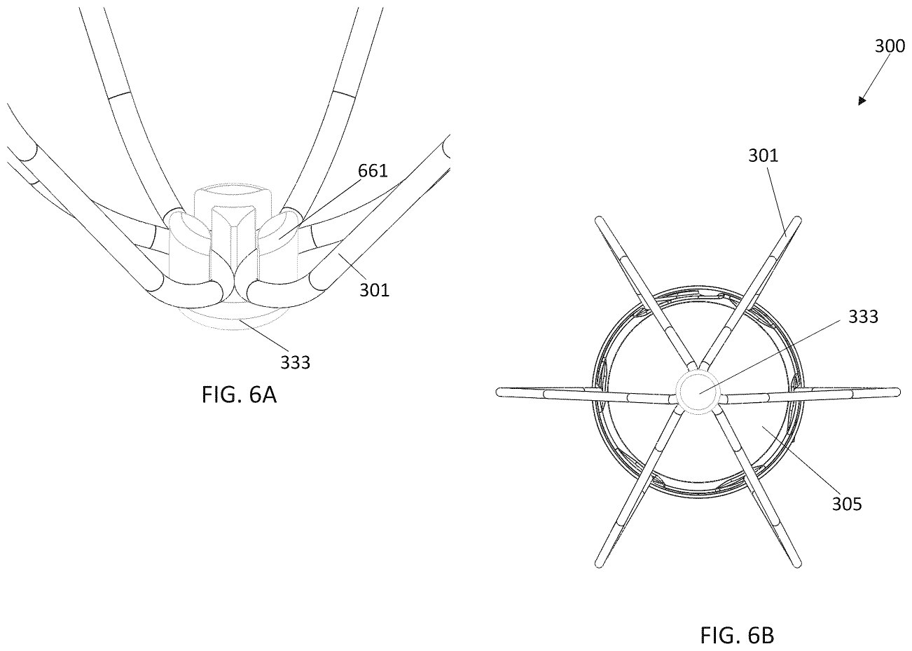

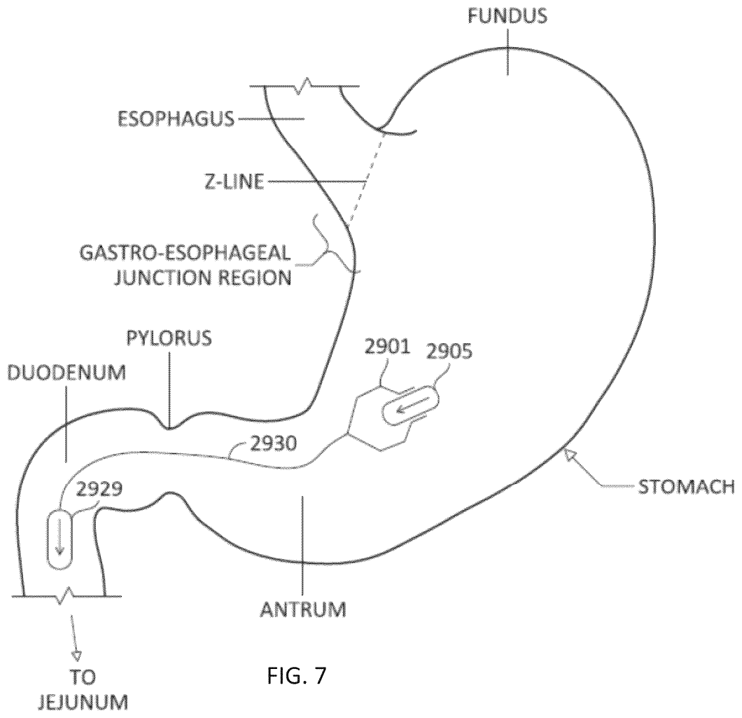

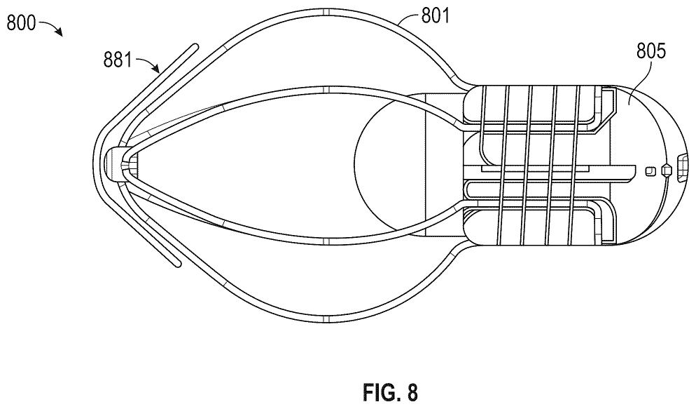

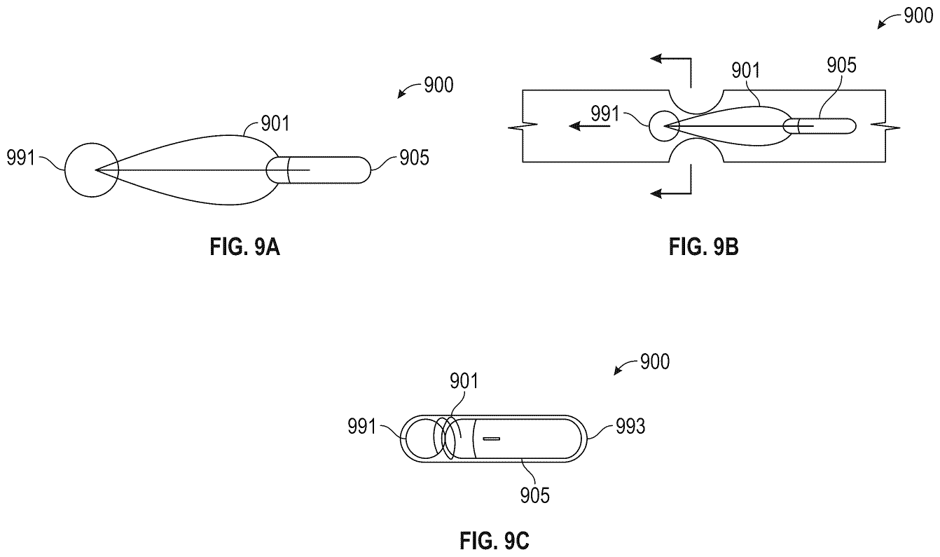

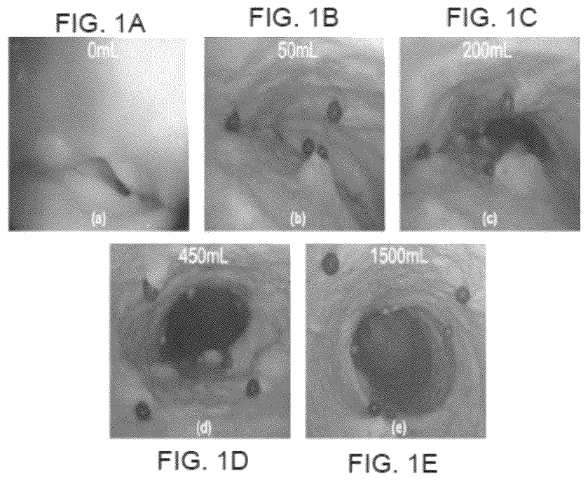

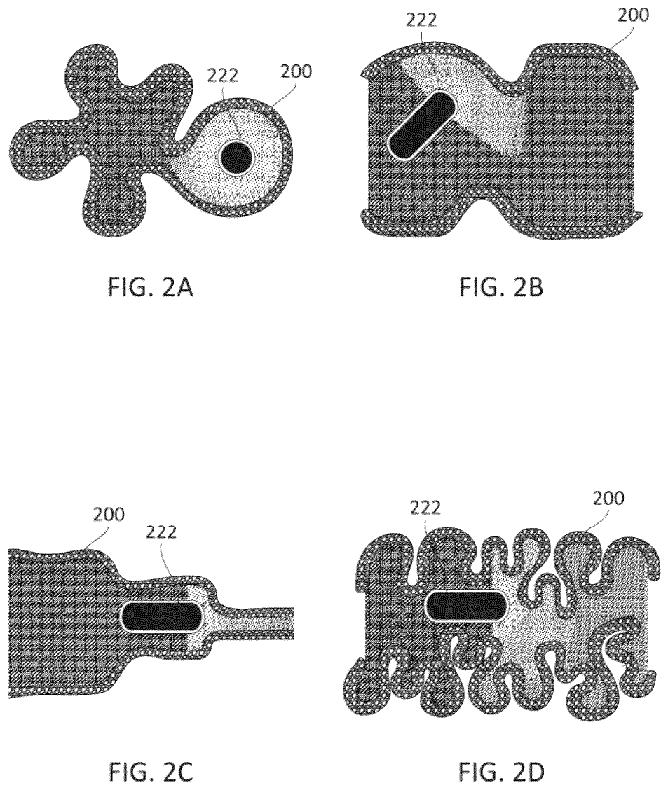

### DETAILED DESCRIPTION

Described herein are devices for use with a capsule endoscope (CE) (e.g., a pill camera, pillcam, wireless capsule endoscope, or video capsule endoscope (VCE)) that significantly aid in more complete luminal visualization during capsule endoscopy. The devices create local distension of gastrointestinal luminal tissue away from the camera, improving diagnostic yield.

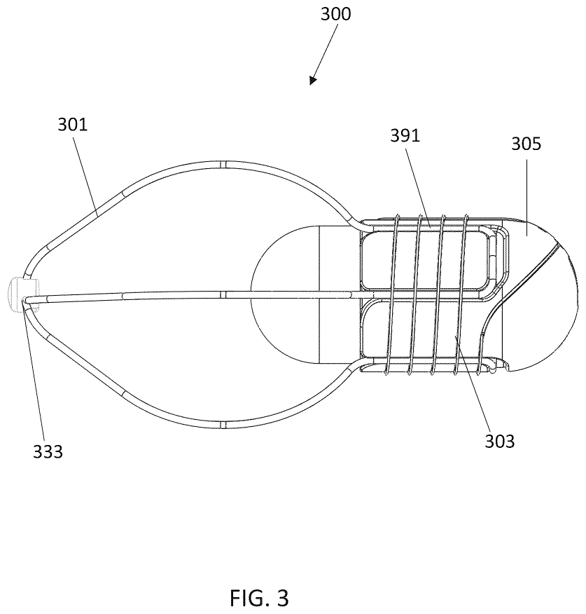

An exemplary luminal expansion device 300 is shown in FIG. 3. The device 300 includes a plurality of radiating struts 301 configured to attach to the capsule endoscope 305 and extend both axially and radially away from the camera lens. The struts 301 can cross or meet at an apex 333, which is positioned a maximum axial distance away from the lens of the capsule endoscope 305. Further, the struts 301 can be attached to the capsule endoscope 305 with a retention strap 303. The retention strap 303 can extend around the proximal ends 391 of the struts 301 and the central portion of the pillcam 305 to hold the struts 301 onto the pillcam 305. For example, the retention strap 303 can be wrapped a plurality of times (e.g., 2-10 times) around the proximal ends 391 and scope 305, e.g., in a spiraling fashion. The retention strap 303 can be, for example, a tensile element such as a suture, string, wire, filament, or braid.

In use, the struts 301 can be configured to distend tissue (e.g., within the small intestine) such that luminal folds are unfolded, enabling more complete visualization. The apex 333 can act as the leading edge while the wedge or conical shape of the tip can open tissue up as the device 300 moves through, maintaining an open field of view for the camera of the capsule endoscope 305. The primarily open structure of the device 300 can allow for direct, unobscured viewing of the tissue and can enable the thru-passage of matter, such as chyme, during use.

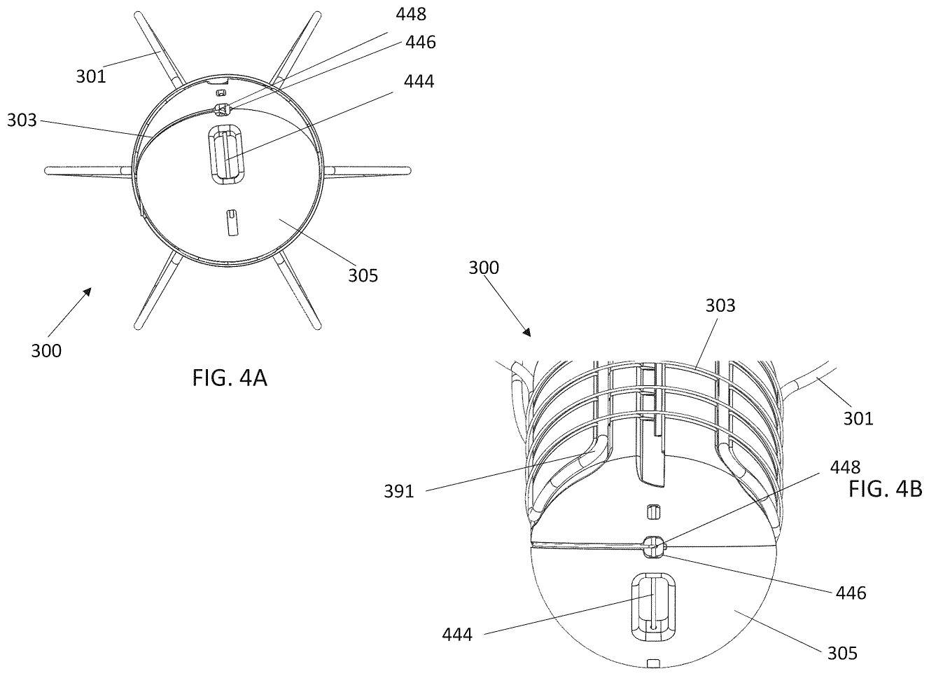

Referring to FIGS. 4A-4B, in some embodiments, the struts 301 can be released from the capsule endoscope 305 with a trigger mechanism, such as a fuse wire 444. Releasing the struts 301 from the capsule endoscope 305 can advantageously provide easier passage through the body after imaging is complete and/or release the capsule endoscope 305 if the struts 301 get caught, for example, in a polyp, stricture, or diverticulum.

The fuse wire 444 can include a section of material that is engineered to degrade with upon activation of an actuator and/or with the passage of time. For example, the fuse wire 444 can be a galvanic time release (GTR) wire. The GTR wire can include plated elements or regions (cathodes) with elements or regions of a highly active material (anode). When the GTR wire is placed in electrolyte-like gastrointestinal fluids, corrosion current between the anode and the cathode can drive electrons within the metal and ions within the electrolyte such that the active material electrochemically degrades, evolving hydrogen gas. In one example, the plating can be a silver plating either through direct deposit onto the wire or an intermediate layer, such as nickel. The silver plating can function as the cathodic portion of the wire. Alternatively, other noble metals can be used as the cathodic portion, including, for example, gold, a gold-platinum alloy, rhodium plated on silver-plated copper, silver, monel metal, or high nickel-copper alloys. The retention strap 303 can be secured around the unplated portion of the fuse wire 444. After the fuse wire 444 corrodes, the wrapped retention strap 303 can come loose and unwrap from the proximal ends 391 of the struts 301 such that the struts 301 release and become independent of the capsule endoscope 305.

In another example, the fuse wire 444 can be a nutritional metal that itself corrodes, such as magnesium. For example, the fuse wire 444 can be made of the magnesium alloy Resoloy®. As the magnesium corrodes, the magnesium can provide a portion of the recommended 420 mg per day provided by the United States Recommended Dietary Allowance. In some embodiments, part of the magnesium wire can then be plated, e.g., with silver, to act as a cathode. The amount of plating can be used to control the fuse time. For example, in some embodiments, substantially all of the wire can be plated except at the connection with the retention strap 303 so as to concentrate corrosion to a small area and minimize the fuse time. In other embodiments, a smaller length of the wire can be plated (e.g., 30-90%, such as 50-60% can be plated) so as to increase fuse time. In yet other embodiments, the plated area of the wire can be covered so as to decrease the reaction rate and increase fuse time.

In some embodiments, the fuse wire 444 can function entirely via corrosion of the materials upon use in the body (e.g., due to interaction of the fuse wire 444 with gastrointestinal fluids). In other embodiments, the fuse wire 444 can be connected to a power supply, such as a battery, to accelerate corrosion. The power supply can be activated, for example, by an external actuator, such as a magnetic actuator, or by an internal actuator in the device.

In another example, the fuse wire 444 can be made of a material that dissolves in the body (e.g., in gastrointestinal fluid), such as a polyvinyl alcohol (e.g., Vinex™ or Aquasol™) or a dissolvable suture material.

In some embodiments, the fuse wire 444 can be inserted through a bore in the capsule endoscope 305 and attached to the capsule endoscope 305 such that the remnants remain in place on the scope 305 after the selective regions of the wire 444 have corroded.

Referring to FIG. 4B, in some embodiments, there can be an aperture or open window 446 that allows gastrointestinal fluid to access the fuse wire 444. The fuse wire 444 can have a diameter, for example, of between 0.003 inches and 0.030 inches, such as around 0.015 inches.

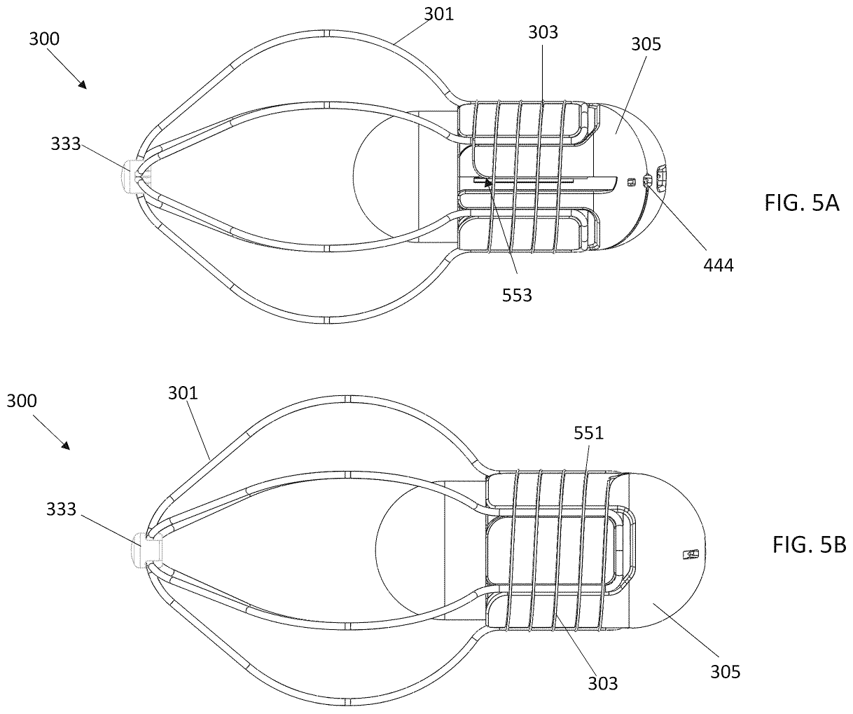

Referring to FIGS. 5A-5B, in some embodiments, the retention strap 303 can be configured to sit within one or more retention strap grooves 551 on an outer surface of the body of the capsule endoscope 305 (e.g., within a groove that spirals around the endoscope 305 a plurality of times) so as to ensure that the retention strap 303 protrudes only minimally radially outwards relative to the capsule endoscope 305 (the struts 301 can also sit within one or more grooves on the body of the endoscope 305). The retention strap 303 can further terminate in a slot 553 on the endoscope capsule 305 that allows the strap 303 to be secured thereto.

In some embodiments, to capture the struts 301 with the retention strap 303, the retention strap 303 can be tied to the fuse 444 (e.g., with a knot 448). The retention strap 303 can then be wrapped around the body of the capsule endoscope 305 and the proximal ends 391 of the struts 301. Further, in some embodiments, a pull-through loop can be used to pull the free end of the retention strap 303 back under all of the wraps of the strap 303 before terminating it in the slot 553.

In some embodiments, rather than having a separate retention strap 303 and fuse wire 444, the fuse wire itself can act as a retention member (e.g., can be configured to wrap around the proximal ends 391 of the struts 301 and the capsule endoscope 305 to hold the struts 301 onto the scope 305). In such an embodiment, the struts 301 can be released from the scope 305 upon failure of the fuse wire.

In some embodiments, the fuse or retention strap failure time can be engineered by optimizing the potential between the anode and cathode when immersed within the gastrointestinal fluid, the surface area of the fuse wire, and the cross-sectional area of the fuse wire. In some embodiments, the fuse or retention strap can be configured to fail at a longer duration than the battery of the capsule endoscope. For example, the fuse can be engineered to fail at approximately 12-15 hours. Having the fuse or retention strap fail after the battery of the capsule endoscope has been depleted can help ensure that the endoscope is safely removed after completion of imaging.

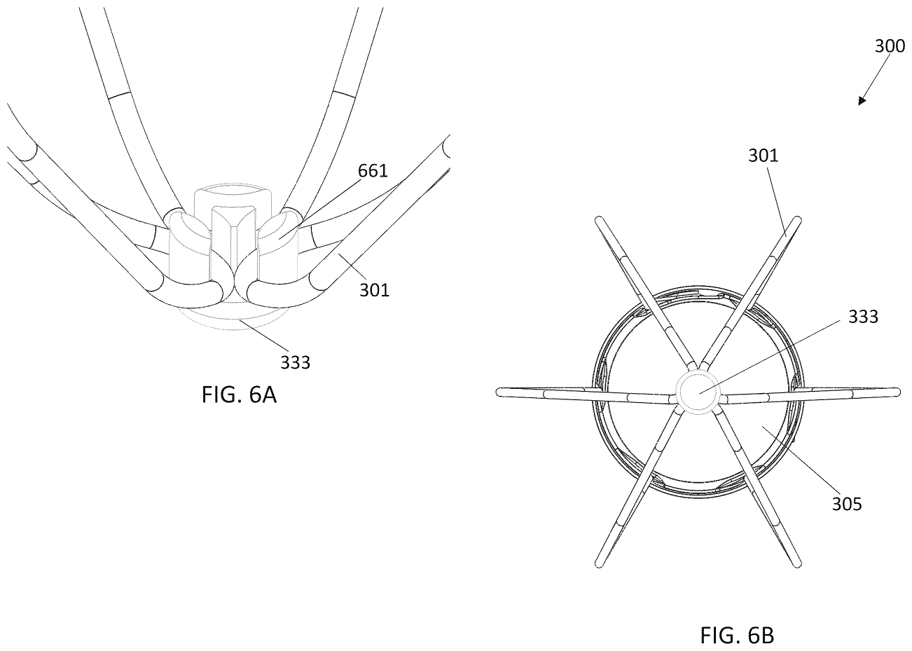

Referring to FIGS. 6A-6B, in some embodiments, the apex 333 can include a plurality of proximally extending pins 661 around which the struts 301 can wind. For example, the neighboring struts can be formed of a single elongate element that is wrapped around the pins 661. In one embodiment and as shown in FIGS. 6A-6B, there can be three pins 661, three elongate loops, and six struts 301. In another embodiment, a pin can be integrated into one of the elongate loops, and mating female hoop geometry can be integrated into the remaining two loops. Advantageously, such designs can thus allow for the struts 301 to separate at least partially from one another when the struts 301 are released from the capsule endoscope 305. In another embodiment, the apex 333 can include a separately corrodible element that releases the struts 301 from one another.

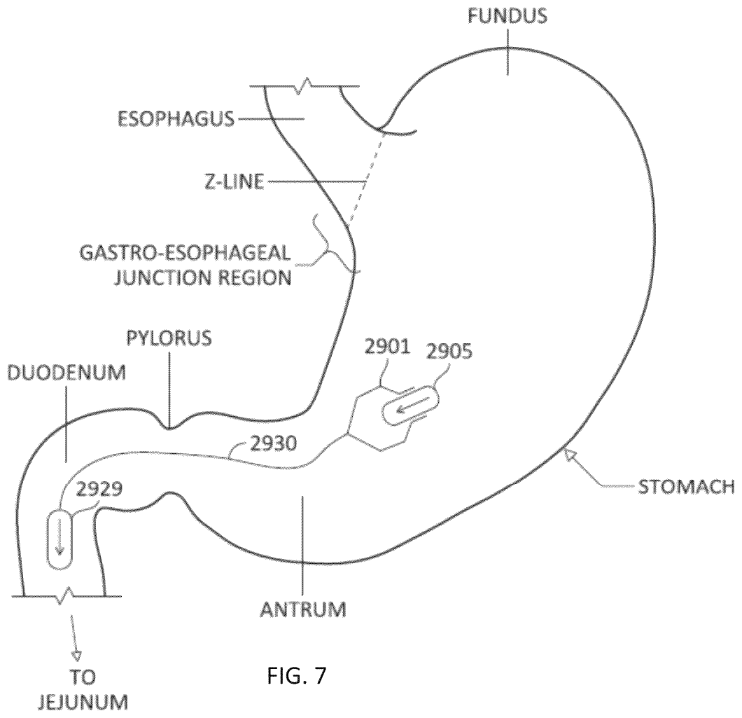

Referring to FIG. 7, in some embodiments, a small tug element 2929 can be attached to the struts 2901 or capsule endoscope 2905 with a tensile member 2930. The small tug element 2929 can thus easily pass through the pylorus and, as it is propelled, pull the struts 2901 and attached capsule endoscope 2905 through the pylorus and into the duodenum. The tug element 2929 can, for example, be manipulated via peristalsis. Alternatively or additionally, the tug element 2929 can be manipulated by external magnetic forces that tug the device. Alternatively, the external magnetic force can act directly on a magnetic element in the luminal expansion device such that the luminal expansion device can be actively pulled through the pylorus without a tug element.

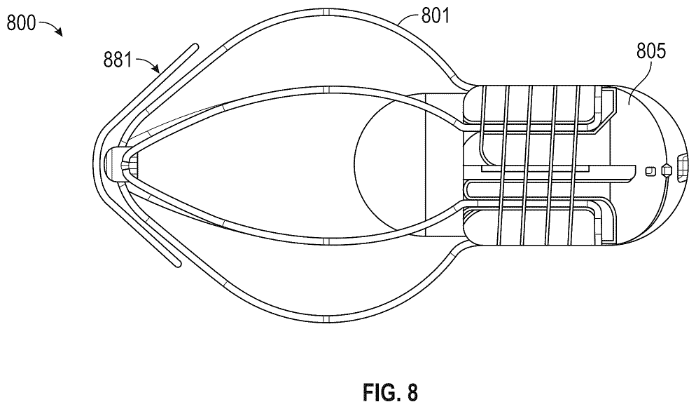

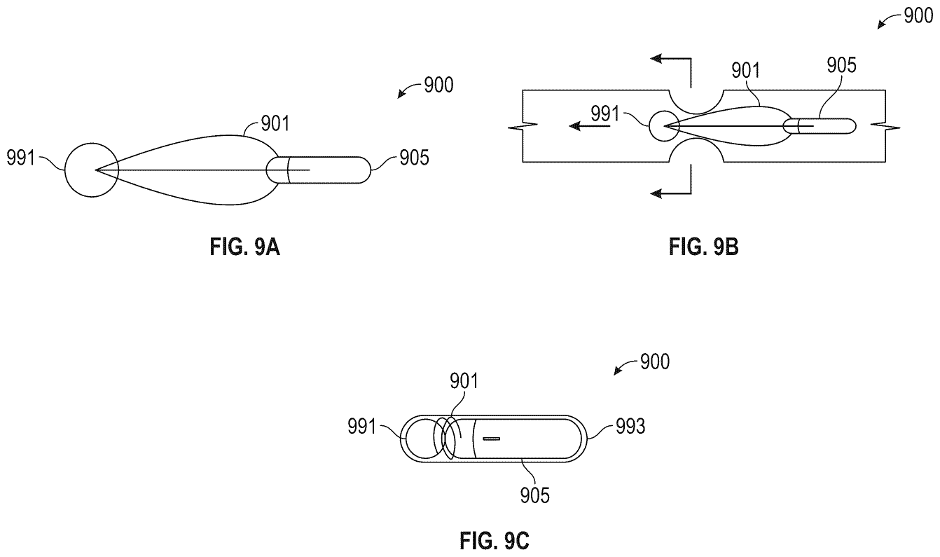

Referring to FIG. 8, in some embodiments, a luminal expansion device 800 can include a curved lens 881 at the apex of the struts 801. The curved lens 881 can be clear (e.g., a clear plastic) to allow for imaging therethrough and can be configured to nest on top of the lens of the capsule endoscope 805 when the device 800 is in the stowed configuration. In some embodiments, the stowed configuration may comprise a gel cap that holds the expansion device 800 in a compact configuration until release (for instance, as shown in FIG. 9C). The curved lens 881 at the distal end of the device 800 can advantageously prevent wedging of the luminal expansion device into the tissue folds during use.

Referring to FIGS. 9A-9C, in some embodiments, a luminal expansion device 900 can include a sphere 991 at the distal apex of the struts 901. The sphere 991 can, for example, be made of a dissolvable material. Further, the sphere 991 can advantageously provide an atraumatic tip to prevent wedging of the luminal expansion device into the tissue folds during use. Additionally, as shown in FIG. 9B, the sphere 991 can assist in peristaltic movement of the device 900 and capsule endoscope 905. Similar to device 800, a dissolvable gel cap 993 can be used to hold the device 900 in the stowed configuration (see FIG. 9C).

In some embodiments, the struts of the luminal expansion devices described herein can be configured to be held in a constrained configuration by a restraining element. In some embodiments, the restraining element can be released by an external magnetic force. This can be triggered, for example, based on the location of the capsule endoscope, such as its passage through the pylorus.

Any of the features or elements of any of the expansion devices described herein may be combined or substituted for features or elements of any other expansion device.

Additional details pertinent to the present invention, including materials and manufacturing techniques, may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are a plurality of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below can be termed a second feature/element, and similarly, a second feature/element discussed below can be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

What is claimed is:

1. A method of enhancing visualization of a body lumen, the method comprising:
positioning a device including a capsule endoscope and a plurality of struts attached thereto into a body lumen;
expanding the plurality of struts of the device within the body lumen such that the plurality of struts extend away from a camera lens of the capsule endoscope and meet at an apex that is positioned a set axial distance away from the camera lens;
imaging with the camera lens into the body lumen; and
using a magnetic force to pull the device through a pylorus.
